(19) United States
(12) Patent Application Publication
(10) Pub. No.: US 2026/0176213 A1
(43) Pub. Date: Jun. 25, 2026

(54) Fluorination Methods of Arenes Using Arylbenziodoxolones and Compositions Related Thereto

(71) Applicant: Emory University, Atlanta, GA (US)

(72) Inventor: Huan Liang, Atlanta, GA (US)

(21) Appl. No.: 19/427,028

(22) Filed: Dec. 19, 2025

Related U.S. Application Data
(60) Provisional application No. 63/736,372, filed on Dec. 19, 2024.

(51) Int. Cl. C07B 59/00; A61K 51/04

(52) U.S. Cl. CPC C07B 59/001; A61K 51/04; C07B 2200/05

(57) ABSTRACT

Disclosed herein are tracer and precursor compounds for performing imaging methods such as positron emission tomography (PET). In certain embodiments, this disclosure relates to methods of forming ¹⁸F substituted aromatic compounds comprising contacting an aryl compound substituted with an aryl[d][1,2]iodaoxol-3-one group and a fluorine ion under conditions providing a fluorine aromatic compound with a fluorine in place of the aryl[d][1,2]iodaoxol-3-one group.

### CROSS-REFERENCE TO RELATED APPLICATIONS

This application claims the benefit of U.S. Provisional Application No. 63/736,372 filed Dec. 19, 2024. The entirety of this application is hereby incorporated by reference for all purposes.

### BACKGROUND

Positron emission tomography (PET) is a medical imaging technique used to investigate biological processes in vivo by detecting signals from exogenous radioactive probes. PET can be used in monitoring disease progression and assessing drug efficacy. PET tracers can include fluorine enriched with isotope 18, also referred to as ¹⁸F. Nucleophilic aromatic substitution with [¹⁸F]fluoride ion is a method for producing ¹⁸F-fluoroarenes.
Tredwell et al. report methods labeling arenes with ¹⁸F. Angew Chem Int Ed Engl, 2012, 51(46):11426-37.
Ozerskaya et al. report the synthesis of 2-fluorobenzoic acids by nucleophilic fluorination of 1-arylbenziodoxolones. Arkivoc, 2022, part vii, 108-125.
Lancer et al. report the ortho effect in the pyrolysis of iodonium halides. J Org Chem, 1976, 41(21): 3360.
Rotstein et al. report spirocyclic hypervalent iodine(III)-mediated radiofluorination of non-activated and hindered aromatics. Nature Communications, 2014, volume 5, Article number: 4365.
Cardinale et al. report using iodonium ylides for radiofluorination of electron rich arenes. RSC Adv., 2014, 4, 17293.

References cited herein are not an admission of prior art.

### SUMMARY

Disclosed herein are tracer and precursor compounds for performing imaging methods such as positron emission tomography (PET). In certain embodiments, this disclosure relates to methods of forming fluorine substituted aromatic compounds comprising contacting an aryl compound substituted with an aryl[d][1,2]iodaoxol-3-one group and a fluorine ion under conditions providing a fluorine aromatic compound with a fluorine in place of the aryl[d][1,2]iodaoxol-3-one group.

In certain embodiments, this disclosure relates to methods for synthesizing ¹⁸F-fluoroarenes through radiofluorination of 1-arylbenziodoxolones, along with approaches to obtain the corresponding labeling precursors from aryl trifluoroborates, aryl silanes, or aryl stannanes.

In certain embodiments, this disclosure relates to methods of forming a fluorinated aromatic compound comprising contacting an aryl compound substituted with an aryl[d][1,2]iodaoxol-3-one group and a fluoro ion under conditions providing a fluorinated aromatic compound with a fluorine in place of the aryl[d][1,2]iodaoxol-3-one group. In certain embodiments, the fluoro ion is isotopically enriched with fluorine 18 isotope, ¹⁸F. In certain embodiments, the aryl[d][1,2]iodaoxol-3-one group is a benzo[d][1,2]iodaoxol-3-one group optionally substituted.

In certain embodiments, said conditions include the presence of a base in a solvent at a temperature above 100 degrees Celsius. In certain embodiments, the base is a quaternary ammonium base. In certain embodiments, the quaternary ammonium base is a tetra-alkyl ammonium salt. In certain embodiments, the tetra-alkyl ammonium salt is tetraethylammonium bromide (TEAB).

In certain embodiments, the solvent is dimethyl sulfoxide (DMSO) or dimethylformamide (DMF).

In certain embodiments, the reaction temperature is above 100 degrees Celsius. In certain embodiments, the reaction temperature is above 120 degrees Celsius. In certain embodiments, the temperature is above 140 degrees Celsius.

In certain embodiments, this disclosure relates to methods of forming an aryl compound substituted with an aryl[d][1,2]iodaoxol-3-one group, e.g., for further use in producing a halogenated, fluorinated, or ¹⁸F aromatic compound, comprising contacting an aryl compound substituted with trialkyltin, trichloroborate, or trialkylsilane and aryl[d][1,2]iodaoxol-3-one substituted at the iodine with a halogen or acetate under conditions such that an aryl compound substituted with an aryl[d][1,2]iodaoxol-3-one group is formed.

In certain embodiments, this disclosure relates to methods of imaging a radionuclide reported herein comprising administering the radionuclide to a subject and detecting the radionuclide at a location or specific area in the subject. In certain embodiments, methods further comprise recording the location(s) of the radionuclide on a non-transitory computer readable medium. In certain embodiments, methods further comprise creating an image based on locations of the radionuclide in the area.

In certain embodiments, this disclosure relates to the compound 2-azidoethyl 4-(3-oxo-1l3-benzo[d][1,2]iodaoxol-1(3H)-yl)benzoate.

In certain embodiments, this disclosure relates to the compound 2-azidoethyl 4-(fluoro-¹⁸F)benzoate.

In certain embodiments, this disclosure relates to the compound N-(2-(2-cyano-4,4-difluoropyrrolidin-1-yl)-2-oxoethyl)-6-(3-(prop-2-yn-1-ylamino)propoxy)quinoline-4-carboxamide or salt thereof.

In certain embodiments, this disclosure relates to the compound 2-(4-(((3-((4-((2-(2-cyano-4,4-difluoropyrrolidin-1-yl)-2-oxoethyl)carbamoyl)quinolin-6-yl)oxy)propyl)amino)methyl)-1H-1,2,3-triazol-1-yl)ethyl 4-(fluoro-¹⁸F)benzoate or salt thereof.

In certain embodiments, this disclosure relates to the compound 3-(3-oxo-1l3-benzo[d][1,2]iodaoxol-1(3H)-yl)-5-(pyridin-2-ylethynyl)benzonitrile or salt thereof.

In certain embodiments, this disclosure relates to the compound 3-(fluoro-¹⁸F)-5-(pyridin-2-ylethynyl)benzonitrile or salt thereof.

In certain embodiments, this disclosure relates to methods of using the radionuclide tracer of 2-(4-(((3-((4-((2-(2-cyano-4,4-difluoropyrrolidin-1-yl)-2-oxoethyl)carbamoyl)quinolin-6-yl)oxy)propyl)amino)methyl)-1H-1,2,3-triazol-1-yl)ethyl 4-(fluoro-¹⁸F)benzoate or 3-(fluoro-¹⁸F)-5-(pyridin-2-ylethynyl)benzonitrile by administering the radionuclide to an area of a subject and detecting the radionuclide at a location or in an area in the subject.

In certain embodiments, the disclosure contemplates methods of imaging a tumor comprising administering a compound disclosed herein with a 2-(4-(((3-((4-((2-(2-cyano-4,4-difluoropyrrolidin-1-yl)-2-oxoethyl)carbamoyl)quinolin-6-yl)oxy)propyl)amino)methyl)-1H-1,2,3-triazol-1-yl)ethyl 4-(fluoro-¹⁸F)benzoate or 3-(fluoro-¹⁸F)-5-(pyridin-2-ylethynyl)benzonitrile tracer to a subject and viewing/imaging the compound by PET.

In certain embodiments, the disclosure contemplates methods of preparing compounds disclosed herein by mixing starting materials under conditions such that the compounds are formed.

In certain embodiments, this disclosure relates to a method of making 3-(fluoro-¹⁸F)-5-(pyridin-2-ylethynyl)benzonitrile comprising contacting 3-(3-oxo-1l3-benzo[d][1,2]iodaoxol-1(3H)-yl)-5-(pyridin-2-ylethynyl)benzonitrile and a fluoro ion under conditions providing 3-(fluoro-¹⁸F)-5-(pyridin-2-ylethynyl)benzonitrile, i.e., a fluorinated aromatic compound with a fluorine in place of the aryl[d][1,2]iodaoxol-3-one group.

In certain embodiments, this disclosure relates to a method of making 2-azidoethyl 4-(fluoro-¹⁸F)benzoate comprising contacting 2-azidoethyl 4-(3-oxo-1l3-benzo[d][1,2]iodaoxol-1(3H)-yl)benzoate and a fluoro ion under conditions providing 2-azidoethyl 4-(fluoro-¹⁸F)benzoate, i.e., a fluorinated aromatic compound with a fluorine in place of the benzo[d][1,2]iodaoxol-3-one group.

In certain embodiments, this disclosure relates to a method of making (S)-2-(4-(((3-((4-((2-(2-cyano-4,4-difluoropyrrolidin-1-yl)-2-oxoethyl)carbamoyl)quinolin-6-yl)oxy)propyl)amino) methyl)-1H-1,2,3-triazol-1-yl)ethyl 4-(fluoro-¹⁸F)benzoate comprising contacting 2-azidoethyl 4-(fluoro-¹⁸F)benzoate and (S)—N-(2-(2-cyano-4,4-difluoropyrrolidin-1-yl)-2-oxoethyl)-6-(3-(prop-2-yn-1-ylamino)propoxy)quinoline-4-carboxamide under conditions providing(S)-2-(4-(((3-((4-((2-(2-cyano-4,4-difluoropyrrolidin-1-yl)-2-oxoethyl)carbamoyl)quinolin-6-yl)oxy)propyl)amino) methyl)-1H-1,2,3-triazol-1-yl)ethyl 4-(fluoro-¹⁸F)benzoate.

In certain embodiments, this disclosure relates to a growth medium comprising 2-(4-(((3-((4-((2-(2-cyano-4,4-difluoropyrrolidin-1-yl)-2-oxoethyl)carbamoyl)quinolin-6-yl)oxy)propyl) amino)methyl)-1H-1,2,3-triazol-1-yl)ethyl 4-(fluoro-¹⁸F)benzoate or 3-(fluoro-¹⁸F)-5-(pyridin-2-ylethynyl)benzonitrile tracer. In certain embodiments, the growth medium further comprises a cancer cell, tumor, or a sample from a patient for determining whether the sample contains a cancerous, neuronal, cardiac, inflamed cell or tissue.

In certain embodiments, methods further comprise recording the location of the radionuclide in the subject on a non-transitory computer readable medium. In certain embodiments, methods further comprise creating an image based on locations of the radionuclide in the area.

In certain embodiments, methods further comprise the step of recording the imaging, detecting, measuring, or quantifying data or diagnostic indications therefrom on a non-transitory computer readable medium. In certain embodiments, methods further comprise the step of reporting the imaging, detecting, measuring, or quantifying data or diagnostic indications therefrom to a medical professional.

### BRIEF DESCRIPTION OF THE SEVERAL VIEWS OF THE DRAWINGS

FIG. 1A illustrates ¹⁸F-fluorination with hypervalent iodine precursors. Some desirable aspects of this method include that the precursors can be prepared in mild and/or non-oxidative conditions, there is broad substrate scope and functional group tolerance, metal free, desirable product yield and suitable parameters for preparing ¹⁸F probes.

FIG. 1B shows the chemical structures and the associated chemical names benzo[d][1,2]iodaoxol-3-one and aryl[d][1,2]iodaoxol-3-one.

FIG. 2A illustrates the synthesis of 1-arylbenziodoxolones from aryl trifluoroborates, aryl silanes, or aryl stannanes.

FIG. 2B shows various conditions (A to F) and yields using alternative metal catalysts, additives, solvents, and temperatures.

FIG. 3A shows a scheme of producing various aryl substituted variants of 1-arylbenziodoxolones using conditions A to F as in FIG. 2B.

FIG. 3B shows synthetic yields using alternative electron donating substituted aryl groups.

FIG. 3C shows synthetic yields using alternative electron withdrawing substituted aryl groups.

FIG. 3D shows synthetic yields using alternative fused and hetero aryl groups.

FIG. 3E shows synthetic yields using alternative oxidative sensitive aryl groups.

FIG. 4A shows ¹⁸F substitution using conditions at various temperatures and solvents.

FIG. 4B illustrates optimized conditions for synthesis of ¹⁸F derivatives.

FIG. 4C shows results using the optimized conditions for electron rich aryls.

FIG. 4D shows results using the optimized conditions for electron deficient aryls.

FIG. 4E shows results using the optimized conditions for fused or hetero aryls.

FIG. 4F shows results using the optimized conditions for oxidation sensitive aryls.

FIG. 5A illustrates ¹⁸F fluorination of 2-azidoethyl 4-(3-oxo-1[lambda]³-benzo[d][1,2]iodaoxol-1(3H)-yl)benzoate. Fluorine 18 (¹⁸F) is substituted in place of the benzo[d][1,2]iodaoxol-3-one group to provide 2-azidoethyl-4-(fluoro-¹⁸F)benzoate.

FIG. 5B illustrates the reaction of 2-azidoethyl-4-(fluoro-¹⁸F)benzoate with compounds having an alkynyl group to ¹⁸F label conjugates by the formation of a triazole linking group, (click reaction) which is further used in imaging methods.

FIG. 5C illustrates ¹⁸F fluorination producing [¹⁸F]FPEB having the chemical name 3-(3-oxo-1[lamda]³-benzo[d][1,2]iodaoxol-1(3H)-yl)-5-(pyridin-2-ylethynyl)benzonitrile. Fluorine 18 (¹⁸F) is substituted in place of the benzo[d][1,2]iodaoxol-3-one group to provide 3-(fluoro-¹⁸F)-5-(pyridin-2-ylethynyl)benzonitrile which is further used in imaging methods.

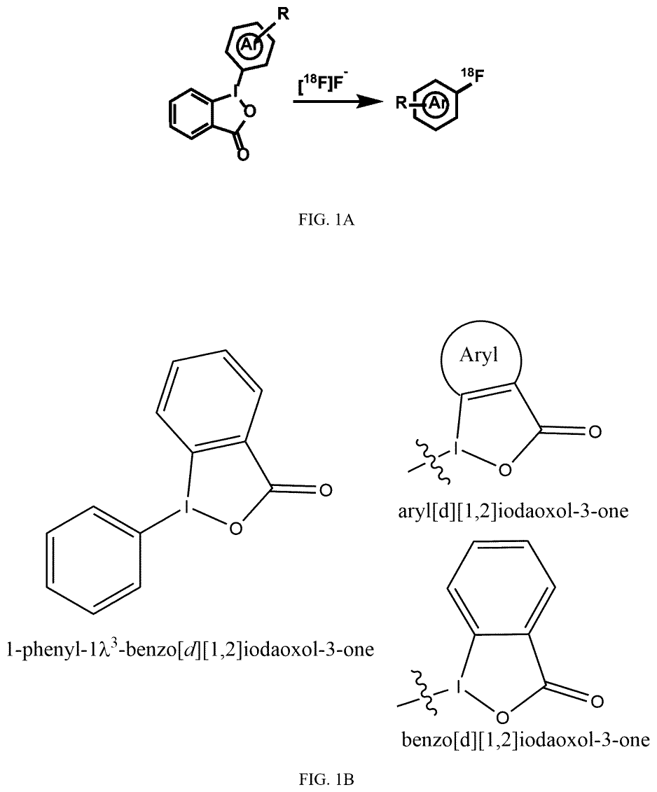

### DETAILED DISCUSSION

Before the present disclosure is described in greater detail, it is to be understood that this disclosure is not limited to particular embodiments described, and as such may, of course, vary. An “embodiment” of this disclosure refers to an example and infers that the example is not necessarily limited to the example. It is also to be understood that the terminology used herein is for the purpose of describing particular embodiments only, and is not intended to be limiting, since the scope of the present disclosure will be limited only by the appended claims or as amended during prosecution.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature. Prior to describing the various embodiments, the following definitions are provided and should be used unless otherwise indicated.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim but may contain additional composition components or method steps, etc., that do not materially affect the basic and novel characteristic(s) of the compositions or methods.

As used herein, “alkyl” means a noncyclic straight chain or branched, unsaturated or saturated hydrocarbon such as those containing from 1 to 10 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Alkylthio” refers to an alkyl group as defined above with the indicated number of carbon atoms attached through a sulfur bridge. An example of an alkylthio is methylthio, (i.e., —S—CH₃).

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above with the indicated number of carbon atoms attached through an amino bridge. An example of an alkylamino is methylamino, (i.e., —NH—CH₃).

“Alkanoyl” refers to an alkyl as defined above with the indicated number of carbon atoms attached through a carbonyl bridge (i.e., —(C═O)alkyl).

“Alkanoyloxy” refers to an alkanoyl attached through an oxygen bridge (i.e., —O—(C═O)alkyl).

“Alkoxycarbonyl” refers to an alkoxy attached through a carboxyl bridge (i.e., —(C═O)—O-alkyl).

“Alkylsulfonyl” refers to an alkyl as defined above with the indicated number of carbon atoms attached through a sulfonyl bridge (i.e., —S(═O)₂alkyl) such as mesyl and the like, and “Arylsulfonyl” refers to an aryl attached through a sulfonyl bridge (i.e., —S(═O)₂aryl).

“Alkylsulfamoyl” refers to an alkyl as defined above with the indicated number of carbon atoms attached through a sulfamoyl bridge (i.e., —NHS(═O)₂alkyl), and an “Arylsulfamoyl” refers to an alkyl attached through a sulfamoyl bridge (i.e., (i.e., —NHS(═O)₂aryl).

“Alkylsulfinyl” refers to an alkyl as defined above with the indicated number of carbon atoms attached through a sulfinyl bridge (i.e. —S(═O)alkyl).

“Azido” refers to the tri-nitrogen group (i.e., —N₃).

“Carbamoyl” refers to a carbonyl attached to an amine group (i.e. —C(═O)NH₂).

“Glycol” refers to an alkyl group substitute with terminal oxygen atoms, and the term is intended to include repeating glycol units, e.g., polyethylene glycol —O(CH₂CH₂O)ₙX, wherein X is typically an H, alkyl or alkanoyl, and n is typically 1-50, 1-100, or 1-1,000.

The terms “halogen” and “halo” refer to fluorine, chlorine, bromine, and iodine.

The term “halogenated alkyl” refers to an alkyl partially or entirely substituted with halogens, e.g., CF₃.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution is optional and therefore it is possible for the designated atom to be unsubstituted.

An unspecified “R” group is a hydrogen, lower alkyl, or aryl all of which may be optionally substituted with one or more substituents. Throughout the specification, groups and substituents thereof may be chosen to provide stable moieties and compounds.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing a oxygen atom with a sulfur atom, replacing an amino group with a hydroxyl group, replacing a nitrogen with a protonated carbon (CH) in an aromatic ring, replacing a bridging amino group (—NH—) with an oxy group (—O—), replacing a bridging amino group (—NH—) with an thio group (—S—), or vice versa. In certain embodiments, a derivative is an alkyl group substituted with a carbocyclyl, e.g., a propyl or butyl group substituted with corresponding cyclopropyl or cyclobutyl group. The derivative may be a prodrug. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

In certain embodiments, the derivative is the substitution of the leaving group, e.g., the acetate group with an alternative leaving group such as chloride, bromide, iodide, nitrate, phosphate, dinitrogen, thioether, phenoxide, sulfonates such as tosylate, mesylate, perfluoroalkyl sulfonates such as triflate or derivatives thereof.

A “linking group” refers to any variety of molecular arrangements that can be used to bridge to molecular moieties together. An example formula may be —Rₙ— wherein R is selected individually and independently at each occurrence as: —CRₙRₙ—, —CHRₙ—, —CH—, —C—, —CH₂—, —C(OH)Rₙ, —C(OH)(OH)—, —C(OH)H, —C(Hal)Rₙ—, —C(Hal)(Hal)-, —C(Hal)H—, —C(N₃)Rₙ—, —C(CN)Rₙ—, —C(CN)(CN)—, —C(CN)H—, —C(N₃)(N₃)—, —C(N₃)H—, —O—, —S—, —N—, —NH—, —NRₙ—, —(C═O)—, —(C═NH)—, —(C═S)—, —(C═CH₂)—, which may contain single, double, or triple bonds individually and independently between the R groups. If an R is branched with an Rₙ it may be terminated with a group such as —CH₃, —H, —CH═CH₂, —CCH, —OH, —SH, —NH₂, —N₃, —CN, or -Hal, or two branched Rs may form a cyclic structure. It is contemplated that in certain instances, each “n” may be individually and independently at each occurrence 1, 2, 3, 4, 5, or 6. It is contemplated that in certain instances, the total Rs or “n” may be less than 100 or 50 or 25 or 10. Examples of linking groups include bridging amide groups, alkyl groups, alkoxy groups, alkoxyalkyl groups, and combinations thereof.

The term, “subject” refers to any animal, preferably a human patient, livestock, e.g., horse, cow, pig, chicken, turkey, mouse, rodent, monkey, or other domestic pet, dog or cat.

“Positron emission tomography” (PET) refers to an imaging technique that produces an image, e.g., three-dimensional image, by detecting pairs of gamma rays emitted indirectly by a positron-emitting radionuclide tracer. Images of tracer concentration within the area are then constructed by computer analysis. A radioactive tracer is administered to a subject e.g., into blood circulation or at a desired location, tissue, or organ. Typically, there is a waiting period while tracer becomes concentrated in areas of interest; then the subject is placed in the imaging scanner. As the radionuclide undergoes positron emission decay, it emits a positron, an antiparticle of the electron with opposite charge, until it decelerates to a point where it can interact with an electron, producing a pair of (gamma) photons moving in approximately opposite directions. These are detected in a scanning device. The technique typically utilizes simultaneous or coincident detection of the pair of photons moving in approximately opposite direction. Photons that do not arrive in pairs (i.e., within a timing-window) are typically ignored. One typically localizes the source of the photons along a straight line of coincidence (also called the line of response, or LOR). This data is used to generate an image.

The term “radionuclide” or “radioactive isotope” refers to molecules of enriched isotopes that exhibit radioactive decay (e.g., emitting positrons). Such isotopes are also referred to in the art as radioisotopes. A radionuclide tracer does not include radioactive primordial nuclides but does include naturally occurring isotopes that exhibit radioactive decay with an isotope distribution that is enriched, i.e., greater than natural abundance. In certain embodiments, it is contemplated that the radionuclides are limited to those with a half live of less than 1 hour and those with a half-life of more than 1 hour but less than 24 hours. Radioactive isotopes are named herein using various commonly used combinations of the name or symbol of the element and its mass number (e.g., ¹⁸F, F-18, or fluorine-18).

Such isotopically labeled compounds are useful in metabolic studies, reaction kinetic studies, detection, or imaging techniques [such as positron emission tomography (PET) or single-photon emission computed tomography (SPECT)] including drug or substrate tissue distribution assays. In particular, an ¹⁸F labeled compound may be particularly preferred for PET or SPECT studies.

One can produce [¹⁸F]fluoride by irradiation of water (containing H₂¹⁸O) with protons resulting in the ¹⁸O(p,n)¹⁸F reaction. The [¹⁸F]isotope is then separated from water and processed for production of a radiopharmaceutical agent. Typically, fluoride recovery is based on ion exchange resins. Typically, the recovery is carried out in two steps (extraction and elution): first the anions (not only fluoride) are separated from the enriched [¹⁸O] water and trapped on a resin and then, said anions, including [¹⁸F]fluoride, are eluted into a mixture containing water, organic solvents, a base, also called activating agent or phase transfer agent or phase transfer catalyst, such as the complex potassium carbonate-Kryptofix 222™ (K₂CO₃—K₂₂₂) or a tetrabutylammonium salt. Kryptofix 222™ is a cyclic crown ether, which binds the potassium ion, preventing the formation of ¹⁸F—KF. Thus, potassium acts as the counter ion of ¹⁸F⁻ to enhance its reactivity but does not interfere with the synthesis. Typical labeling methods use low water content solutions. An evaporation step may follow the recovery of the [¹⁸F]fluoride, e.g., azeotropic evaporation of acetonitrile or other low boiling temperature organic solvent.

Alternatively, the extraction process is performed by passing the [¹⁸F] aqueous solution on a solid support as reported in U.S. Pat. No. 8,641,903. This solid support is typically loaded with a trapping agent, e.g., compound comprising a quaternary amine that is adsorbed on the solid support and allows the [¹⁸F] activity to be trapped because of its positive charge. The solid support is then flushed with a gas or a neutral solvent to remove or push out most of the residual water. The [¹⁸F] is eluted in an organic solvent or in a mixture of organic solvents and is usable for labelling of precursor compounds.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

The cancer to be imaged and/or treated in the context of the present disclosure may be any type of cancer or tumor. These tumors or cancer include, and are not limited to, tumors of the hematopoietic and lymphoid tissues or hematopoietic and lymphoid malignancies, tumors that affect the blood, bone marrow, lymph, and lymphatic system. Hematological malignancies may derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes and myeloproliferative diseases are myeloid in origin.

Also contemplated are malignancies located in the colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, thorax and, more particularly, childhood acute lymphoblastic leukemia, acute lymphoblastic leukemia, acute lymphocytic leukemia, acute myeloid leukemia, adrenocortical carcinoma, adult (primary) hepatocellular cancer, adult (primary) liver cancer, adult acute lymphocytic leukemia, adult acute myeloid leukemia, adult Hodgkin's disease, adult Hodgkin's lymphoma, adult lymphocytic leukemia, adult non-Hodgkin's lymphoma, adult primary liver cancer, adult soft tissue sarcoma, AIDS-related lymphoma, AIDS-related malignant tumors, anal cancer, astrocytoma, cancer of the biliary tract, cancer of the bladder, bone cancer, brain stem glioma, brain tumors, breast cancer, cancer of the renal pelvis and ureter, primary central nervous system lymphoma, central nervous system lymphoma, cerebellar astrocytoma, brain astrocytoma, cancer of the cervix, childhood (primary) hepatocellular cancer, childhood (primary) liver cancer, childhood acute lymphoblastic leukemia, childhood acute myeloid leukemia, childhood brain stem glioma, childhood cerebellar astrocytoma, childhood brain astrocytoma, childhood extracranial germ cell tumors, childhood Hodgkin's disease, childhood Hodgkin's lymphoma, childhood visual pathway and hypothalamic glioma, childhood lymphoblastic leukemia, childhood medulloblastoma, childhood non-Hodgkin's lymphoma, childhood supratentorial primitive neuroectodermal and pineal tumors, childhood primary liver cancer, childhood rhabdomyosarcoma, childhood soft tissue sarcoma, childhood visual pathway and hypothalamic glioma, chronic lymphocytic leukemia, chronic myeloid leukemia, cancer of the colon, cutaneous T-cell lymphoma, endocrine pancreatic islet cells carcinoma, endometrial cancer, ependymoma, epithelial cancer, cancer of the esophagus, Ewing's sarcoma and related tumors, cancer of the exocrine pancreas, extracranial germ cell tumor, extragonadal germ cell tumor, extrahepatic biliary tract cancer, cancer of the eye, breast cancer in women, Gaucher's disease, cancer of the gallbladder, gastric cancer, gastrointestinal carcinoid tumor, gastrointestinal tumors, germ cell tumors, gestational trophoblastic tumor, head and neck cancer, hepatocellular cancer, Hodgkin's disease, Hodgkin's lymphoma, hypergammaglobulinemia, hypopharyngeal cancer, intestinal cancers, intraocular melanoma, islet cell carcinoma, islet cell pancreatic cancer, Kaposi's sarcoma, cancer of kidney, cancer of the larynx, cancer of the lip and mouth, cancer of the liver, cancer of the lung, lymphoproliferative disorders, macroglobulinemia, breast cancer in men, malignant mesothelioma, malignant thymoma, medulloblastoma, melanoma, mesothelioma, occult primary metastatic squamous neck cancer, primary metastatic squamous neck cancer, metastatic squamous neck cancer, multiple myeloma, multiple myeloma/plasmatic cell neoplasia, myelodysplastic syndrome, myelogenous leukemia, myeloid leukemia, myeloproliferative disorders, paranasal sinus and nasal cavity cancer, nasopharyngeal cancer, neuroblastoma, non-Hodgkin's lymphoma during pregnancy, non-melanoma skin cancer, non-small cell lung cancer, metastatic squamous neck cancer with occult primary, buccopharyngeal cancer, malignant fibrous histiocytoma, malignant fibrous osteosarcoma/histiocytoma of the bone, epithelial ovarian cancer, ovarian germ cell tumor, ovarian low malignant potential tumor, pancreatic cancer, paraproteinemias, purpura, parathyroid cancer, cancer of the penis, phaeochromocytoma, hypophysis tumor, neoplasia of plasmatic cells/multiple myeloma, primary central nervous system lymphoma, primary liver cancer, prostate cancer, rectal cancer, renal cell cancer, cancer of the renal pelvis and ureter, retinoblastoma, rhabdomyosarcoma, cancer of the salivary glands, sarcoidosis, sarcomas, skin cancer, small cell lung cancer, small intestine cancer, soft tissue sarcoma, squamous neck cancer, stomach cancer, pineal and supratentorial primitive neuroectodermal tumors, T-cell lymphoma, testicular cancer, thymoma, thyroid cancer, transitional cell cancer of the renal pelvis and ureter, transitional renal pelvis and ureter cancer, trophoblastic tumors, cell cancer of the renal pelvis and ureter, cancer of the urethra, cancer of the uterus, uterine sarcoma, vaginal cancer, optic pathway and hypothalamic glioma, cancer of the vulva, Waldenstrom's macroglobulinemia, Wilms' tumor and any other hyperproliferative disease, as well as neoplasia, located in the system of a previously mentioned organ.

As used herein, the term “neurodegenerative disorder” refers to a disease or condition in which the function of a nervous system of a subject becomes impaired. Examples of contemplated neurodegenerative diseases include Alzheimer's disease, Alexander's disease, Alper's disease, amyotrophic lateral sclerosis, ataxia telangiectasia, Batten disease, bovine spongiform encephalopathy (BSE), chronic fatigue syndrome, corticobasal degeneration, Creutzfeldt-Jakob disease, frontotemporal dementia, Gerstmann-Sträussler-Scheinker syndrome, Huntington's disease, HIV-associated dementia, Kennedy's disease, Krabbe's disease, Lewy body dementia, Machado-Joseph disease, multiple sclerosis, multiple system atrophy, narcolepsy, neuroborreliosis, Parkinson's disease, Pelizaeus-Merzbacher Disease, Pick's disease, primary lateral sclerosis, Refsum's disease, Sandhoff s disease, Schilder's disease, schizophrenia, spinocerebellar ataxia, spinal muscular atrophy, Steele-Richardson-Olszewski disease, or progressive supranuclear palsy.

The terms, “cell culture” or “growth medium” or “media” refers to a composition that contains components that facilitate cell maintenance and growth through protein biosynthesis, such as vitamins, amino acids, inorganic salts, a buffer, and a fuel, e.g., acetate, succinate, a saccharide/disaccharide/polysaccharide, medium chain fatty acids, and/or optionally nucleotides. Typical components in a growth medium include amino acids (histidine, isoleucine, leucine, lysine, methionine, phenylalanine, threonine, tryptophan, valine, and others); vitamins such as retinol, carotene, thiamine, riboflavin, niacin, biotin, folate, and ascorbic acid; carbohydrate such as glucose, galactose, fructose, or maltose; inorganic salts such as sodium, calcium, iron, potassium, magnesium, zinc; serum; and buffering agents. Additionally, a growth medium may contain a pH indicator, e.g., phenol red. Components in the growth medium may be derived from blood serum or the growth medium may be serum-free. The growth medium may optionally be supplemented with albumin, lipids, insulin and/or zinc, transferrin or iron, selenium, ascorbic acid, and an antioxidant such as glutathione, 2-mercaptoethanol or 1-thioglycerol. Other contemplated components contemplated in a growth medium include ammonium metavanadate, cupric sulfate, manganous chloride, ethanolamine, and sodium pyruvate.

Various growth mediums are known in the art. For example, Minimal Essential Medium (MEM) is a term of art referring to a growth medium that contains calcium chloride, potassium chloride, magnesium sulfate, sodium chloride, sodium phosphate and sodium bicarbonate, essential amino acids, and vitamins: thiamine (vitamin B1), riboflavin (vitamin B2), nicotinamide (vitamin B3), pantothenic acid (vitamin B5), pyridoxine (vitamin B6), folic acid (vitamin M), choline, and inositol (originally known as vitamin B8). Dulbecco's modified Eagle's medium (DMEM) is a growth medium which contains additional components such as glycine, serine, and ferric nitrate with increased amounts of vitamins, amino acids, and glucose. Animal serum such as fetal bovine serum (FBS) is sometimes added to a growth media as a supplement.

Certain of the compounds described herein may contain one or more asymmetric centers and may give rise to enantiomers, diastereomers, and other stereoisomeric forms that can be defined, in terms of absolute stereochemistry at each asymmetric atom, as (R)- or (S)- or in terms of the ability to bend plan polarized light in the positive or negative direction. The present chemical entities, compositions and methods are meant to include all such possible isomers, including racemic mixtures, tautomer forms, hydrated forms, optically substantially pure forms, and intermediate mixtures.

In certain embodiments, this disclosure relates to methods of making compounds disclosed herein comprising contacting a starting material with reagents reported herein under conditions that the products are formed as reported herein. In certain embodiments, methods further comprise purifying and isolating the desired product e.g., by recrystallization or chromatographic methods. In certain embodiments, this disclosure relates to a compound disclosed herein with greater than 70%, 80%, 90%, 95%, of 97% purity by weight. In certain embodiments, if the product has a chiral center, the product may be purified to greater than 70%, 80%, 90%, 95%, or 97% enantiomeric excess or diastereomeric excess.

### Producing a Fluorinated Aromatic Compound or Radionuclide Thereof

In certain embodiments, this disclosure relates to methods for synthesizing ¹⁸F-fluoroarenes through radiofluorination of 1-arylbenziodoxolones, along with approaches to obtain the corresponding labeling precursors from aryl trifluoroborates, aryl silanes, or aryl stannanes.

In certain embodiments, this disclosure relates to methods of forming a fluorinated aromatic compound comprising contacting an aryl compound substituted with an aryl[d][1,2]iodaoxol-3-one group and a fluoro ion under conditions providing a fluorinated aromatic compound with a fluorine in place of the aryl[d][1,2]iodaoxol-3-one group. In certain embodiments, the fluoro ion is isotopically enriched with fluorine 18 isotope.

In certain embodiments, the aryl[d][1,2]iodaoxol-3-one group is a benzo[d][1,2]iodaoxol-3-one group optionally substituted.

In certain embodiments, said conditions include the presence of a base in a solvent at a temperature above 100 degrees Celsius.

In certain embodiments, the base is a quaternary ammonium base. In certain embodiments, the quaternary ammonium base is a tetra-alkyl ammonium salt. In certain embodiments, the tetra-alkyl ammonium salt is tetraethylammonium bromide (TEAB).

In certain embodiments, the solvent is dimethyl sulfoxide (DMSO) or dimethylformamide (DMF).

In certain embodiments, the temperature is above 100 degrees Celsius. In certain embodiments, the temperature is above 120 degrees Celsius. In certain embodiments, the temperature is above 140 degrees Celsius.

In certain embodiments, the aryl compound substituted with an aryl[d][1,2]iodaoxol-3-one group is selected from following Formula I:

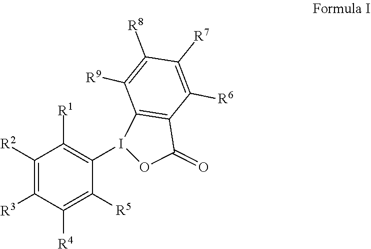

or salt thereof, wherein,
R¹, R², R³, R⁴, R⁵, R⁶, R⁷, R⁸, or R⁹ are, individually and independently, hydrogen, alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹, R², R³, R⁴, R⁵, R⁶, R⁷, R⁸, or R⁹ are optionally substituted with one or more, the same or different, R¹⁰ groups; or
R¹ and R² come together to form a carbocyclyl, aryl, or heterocyclyl optionally substituted with one or more, the same or different, R¹⁰ groups; or
R² and R³ come together to form a carbocyclyl, aryl, or heterocyclyl optionally substituted with one or more, the same or different, R¹⁰ groups; or
R³ and R⁴ come together to form a carbocyclyl, aryl, or heterocyclyl optionally substituted with one or more, the same or different, R¹⁰ groups; or
R⁴ and R come together to form a carbocyclyl, aryl, or heterocyclyl optionally substituted with one or more, the same or different, R¹⁰ groups; or
R⁶ and R⁷ come together to form a carbocyclyl, aryl, or heterocyclyl optionally substituted with one or more, the same or different, R¹⁰ groups; or
R⁷ and R⁸ come together to form a carbocyclyl, aryl, or heterocyclyl optionally substituted with one or more, the same or different, R¹⁰ groups; or
R⁸ and R⁹ come together to form a carbocyclyl, aryl, or heterocyclyl optionally substituted with one or more, the same or different, R¹⁰ groups;
R¹⁰ is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹⁰ is optionally substituted with one or more, the same or different, R¹¹ groups;
R¹¹ is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹¹ is optionally substituted with one or more, the same or different, R¹² groups;
R¹² is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹² is optionally substituted with one or more, the same or different, R¹³ groups;
R¹³ is halogen, cyano, hydroxy, trifluoromethoxy, trifluoromethyl, amino, azido, nitro, formyl, carboxy, carbamoyl, mercapto, methyl, ethyl, ethynyl, methoxy, ethoxy, alkanoyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, the aryl compound substituted with an aryl[d][1,2]iodaoxol-3-one group is selected from following Formula II:

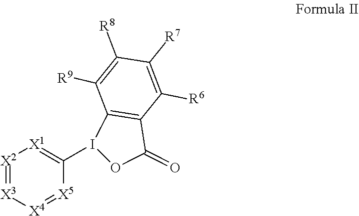

wherein,
X¹ is N or CR¹;
X² is N or CR²;
X³ is N or CR³;
X⁴, is N or CR⁴;
X⁵ is N or CR⁵;
R¹, R², R³, R⁴, R⁵, R⁶, R⁷, R⁸, or R⁹ are, individually and independently, hydrogen, alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹, R², R³, R⁴, R⁵, R⁶, R⁷, R⁸, or R⁹ are optionally substituted with one or more, the same or different, R¹⁰ groups; or
R¹ and R² come together to form a carbocyclyl, aryl, or heterocyclyl optionally substituted with one or more, the same or different, R¹⁰ groups; or
R² and R³ come together to form a carbocyclyl, aryl, or heterocyclyl optionally substituted with one or more, the same or different, R¹⁰ groups; or
R³ and R⁴ come together to form a carbocyclyl, aryl, or heterocyclyl optionally substituted with one or more, the same or different, R¹⁰ groups; or
R⁴ and R⁵ come together to form a carbocyclyl, aryl, or heterocyclyl optionally substituted with one or more, the same or different, R¹⁰ groups; or
R⁶ and R⁷ come together to form a carbocyclyl, aryl, or heterocyclyl optionally substituted with one or more, the same or different, R¹⁰ groups; or
R⁷ and R⁸ come together to form a carbocyclyl, aryl, or heterocyclyl optionally substituted with one or more, the same or different, R¹⁰ groups; or
R⁸ and R⁹ come together to form a carbocyclyl, aryl, or heterocyclyl optionally substituted with one or more, the same or different, R¹⁰ groups;
R¹⁰ is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹⁰ is optionally substituted with one or more, the same or different, R¹¹ groups;
R¹¹ is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹¹ is optionally substituted with one or more, the same or different, R¹² groups;
R¹² is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹² is optionally substituted with one or more, the same or different, R¹³ groups;
R¹³ is halogen, cyano, hydroxy, trifluoromethoxy, trifluoromethyl, amino, azido, nitro, formyl, carboxy, carbamoyl, mercapto, methyl, ethyl, ethynyl, methoxy, ethoxy, alkanoyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, the aryl compound substituted with an aryl[d][1,2]iodaoxol-3-one group is selected from following Formula III:

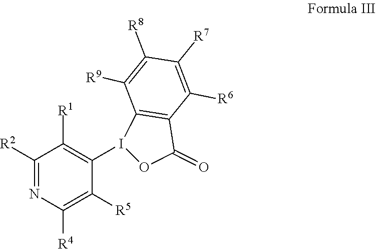

wherein,
R¹, R², R⁴, R⁵, R⁶, R⁷, R⁸, or R⁹ are, individually and independently, hydrogen, alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹, R², R⁴, R⁵, R⁶, R⁷, R⁸, or R⁹ are optionally substituted with one or more, the same or different, R¹⁰ groups; or
R¹ and R² come together to form a carbocyclyl, aryl, or heterocyclyl optionally substituted with one or more, the same or different, R¹⁰ groups; or
R⁴ and R⁵ come together to form a carbocyclyl, aryl, or heterocyclyl optionally substituted with one or more, the same or different, R¹⁰ groups; or
R⁶ and R⁷ come together to form a carbocyclyl, aryl, or heterocyclyl optionally substituted with one or more, the same or different, R¹⁰ groups; or
R⁷ and R⁸ come together to form a carbocyclyl, aryl, or heterocyclyl optionally substituted with one or more, the same or different, R¹⁰ groups; or
R⁸ and R⁹ come together to form a carbocyclyl, aryl, or heterocyclyl optionally substituted with one or more, the same or different, R¹⁰ groups;
R¹⁰ is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹⁰ is optionally substituted with one or more, the same or different, R¹¹ groups;
R¹¹ is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹¹ is optionally substituted with one or more, the same or different, R¹² groups;
R¹² is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹² is optionally substituted with one or more, the same or different, R¹³ groups;
R¹³ is halogen, cyano, hydroxy, trifluoromethoxy, trifluoromethyl, amino, azido, nitro, formyl, carboxy, carbamoyl, mercapto, methyl, ethyl, ethynyl, methoxy, ethoxy, alkanoyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, the aryl compound substituted with an aryl[d][1,2]iodaoxol-3-one group is selected from following Formula IV:

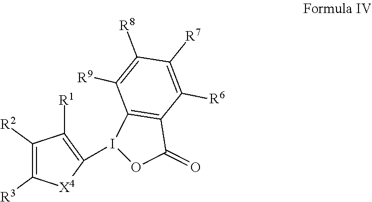

wherein,
X⁴ is O or NR⁴;
R¹, R², R³, R⁴, R⁶, R⁷, R⁸, or R⁹ are, individually and independently, hydrogen, alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹, R², R³, R⁴, R⁶, R⁷, R⁸, or R⁹ are optionally substituted with one or more, the same or different, R¹⁰ groups; or
R¹ and R² come together to form a carbocyclyl, aryl, or heterocyclyl optionally substituted with one or more, the same or different, R¹⁰ groups; or
R² and R³ come together to form a carbocyclyl, aryl, or heterocyclyl optionally substituted with one or more, the same or different, R¹⁰ groups; or
R³ and R⁴ come together to form a carbocyclyl, aryl, or heterocyclyl optionally substituted with one or more, the same or different, R¹⁰ groups; or
R⁶ and R⁷ come together to form a carbocyclyl, aryl, or heterocyclyl optionally substituted with one or more, the same or different, R¹⁰ groups; or
R⁷ and R⁸ come together to form a carbocyclyl, aryl, or heterocyclyl optionally substituted with one or more, the same or different, R¹⁰ groups; or
R⁸ and R⁹ come together to form a carbocyclyl, aryl, or heterocyclyl optionally substituted with one or more, the same or different, R¹⁰ groups;
R¹⁰ is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹⁰ is optionally substituted with one or more, the same or different, R¹¹ groups;
R¹¹ is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹¹ is optionally substituted with one or more, the same or different, R¹² groups;
R¹² is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹² is optionally substituted with one or more, the same or different, R¹³ groups;
R¹³ is halogen, cyano, hydroxy, trifluoromethoxy, trifluoromethyl, amino, azido, nitro, formyl, carboxy, carbamoyl, mercapto, methyl, ethyl, ethynyl, methoxy, ethoxy, alkanoyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, the aryl compound substituted with an aryl[d][1,2]iodaoxol-3-one group is selected from following Formula V:

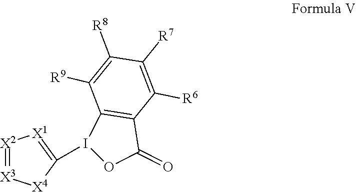

wherein,
X¹ is N or CR¹;
X² is N or CR²;
X³ is N or CR³;
X⁴, is O, NR⁴;
R¹, R², R³, R⁴, R⁶, R⁷, R⁸, or R⁹ are, individually and independently, hydrogen, alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹, R², R³, R⁴, R⁶, R⁷, R⁸, or R⁹ are optionally substituted with one or more, the same or different, R¹⁰ groups; or
R¹ and R² come together to form a carbocyclyl, aryl, or heterocyclyl optionally substituted with one or more, the same or different, R¹⁰ groups; or
R² and R³ come together to form a carbocyclyl, aryl, or heterocyclyl optionally substituted with one or more, the same or different, R¹⁰ groups; or
R³ and R⁴ come together to form a carbocyclyl, aryl, or heterocyclyl optionally substituted with one or more, the same or different, R¹⁰ groups; or
R⁶ and R⁷ come together to form a carbocyclyl, aryl, or heterocyclyl optionally substituted with one or more, the same or different, R¹⁰ groups; or
R⁷ and R⁸ come together to form a carbocyclyl, aryl, or heterocyclyl optionally substituted with one or more, the same or different, R¹⁰ groups; or
R⁸ and R⁹ come together to form a carbocyclyl, aryl, or heterocyclyl optionally substituted with one or more, the same or different, R¹⁰ groups;
R¹⁰ is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹⁰ is optionally substituted with one or more, the same or different, R¹¹ groups;
R¹¹ is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹¹ is optionally substituted with one or more, the same or different, R¹² groups;
R¹² is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹² is optionally substituted with one or more, the same or different, R¹³ groups;
R¹³ is halogen, cyano, hydroxy, trifluoromethoxy, trifluoromethyl, amino, azido, nitro, formyl, carboxy, carbamoyl, mercapto, methyl, ethyl, ethynyl, methoxy, ethoxy, alkanoyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.
   ¹⁸F Aryl with Azido, Alkynyl, or Triazole Linking Groups

In certain embodiments, the ¹⁸F aryl is selected from following formula:

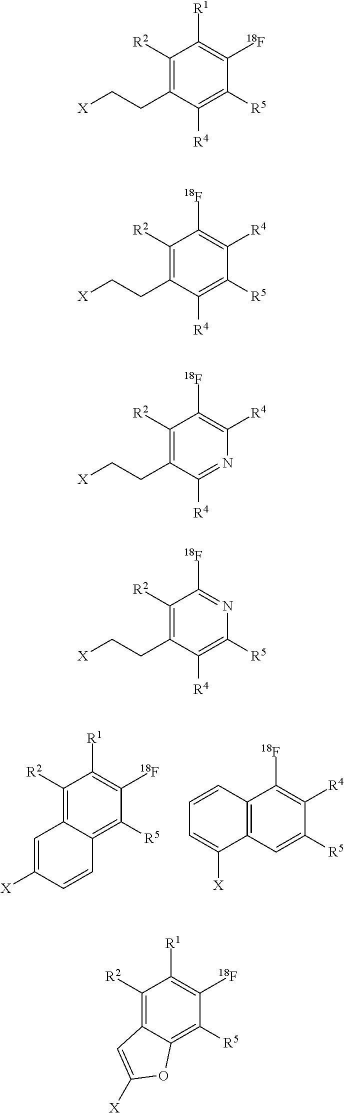

or salt thereof, wherein,
X is alkyl, azido, alkynyl, 1,2,3-triazole, or other linking group conjugated to an antibody, peptide, nucleic acid, or small molecule drug,
R¹, R², R⁴, and R⁵ are, individually and independently, hydrogen, alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹, R², R⁴, and R⁵ are optionally substituted with one or more, the same or different, R¹⁰ groups; or
R¹ and R² come together to form a carbocyclyl, aryl, or heterocyclyl optionally substituted with one or more, the same or different, R¹⁰ groups; or
R⁴ and R⁵ come together to form a carbocyclyl, aryl, or heterocyclyl optionally substituted with one or more, the same or different, R¹⁰ groups; or
R¹⁰ is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹⁰ is optionally substituted with one or more, the same or different, R¹¹ groups;
R¹¹ is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹¹ is optionally substituted with one or more, the same or different, R¹² groups;
R¹² is alkyl, alkenyl, alkynyl, halogen, haloalkyl, cyano, hydroxy, amino, azido, nitro, mercapto, formyl, carboxy, carbamoyl, alkoxy, glycol, alkylthio, alkylamino, (alkyl)₂amino, alkanoyl, alkanoyloxy, alkoxycarbonyl, alkylsulfinyl, alkylsulfonyl, arylsulfonyl, carbocyclyl, aryl, or heterocyclyl, wherein R¹² is optionally substituted with one or more, the same or different, R¹³ groups;
R¹³ is halogen, cyano, hydroxy, trifluoromethoxy, trifluoromethyl, amino, azido, nitro, formyl, carboxy, carbamoyl, mercapto, methyl, ethyl, ethynyl, methoxy, ethoxy, alkanoyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

### Preparation Methods

In certain embodiments, this disclosure relates to methods of forming an aryl compound substituted with an aryl[d][1,2]iodaoxol-3-one group, e.g., for further use in producing a halogenated, fluorinated, or ¹⁸F aromatic compound, comprising contacting an aryl compound substituted with trialkyltin, trichloroborate, or trialkylsilane, and aryl[d][1,2]iodaoxol-3-one substituted at the iodine with a halogen or acetate under conditions such that an aryl compound substituted with an aryl[d][1,2]iodaoxol-3-one group is formed.

In certain embodiments, this disclosure relates to methods of forming an aryl compound substituted with an aryl[d][1,2]iodaoxol-3-one group, e.g., for further use in producing a halogenated, fluorinated, or ¹⁸F aromatic compound, comprising contacting an aryl compound substituted with trialkyltin and aryl[d][1,2]iodaoxol-3-one substituted at the iodine with a halogen or acetate under conditions such that an aryl compound substituted with an aryl[d][1,2]iodaoxol-3-one group is formed.

In certain embodiments, contacting an aryl compound substituted with trialkyltin and aryl[d][1,2]iodaoxol-3-one substituted at the iodine further comprises a silver salt. In certain embodiments, the silver salt is silver nitrate. In certain embodiments, contacting an aryl compound substituted with trialkyltin and aryl[d][1,2]iodaoxol-3-one substituted at the iodine is in an acetonitrile solvent. In certain embodiments, the contacting an aryl compound substituted with trialkyltin and aryl[d][1,2]iodaoxol-3-one substituted at the iodine is at a temperature of less than 100 degrees Celsius.

In certain embodiments, this disclosure relates to methods of forming an aryl compound substituted with an aryl[d][1,2]iodaoxol-3-one group, e.g., for further use in producing a halogenated, fluorinated, or ¹⁸F aromatic compound, comprising contacting an aryl compound substituted with trifluoroborate and aryl[d][1,2]iodaoxol-3-one substituted at the iodine with a halogen or acetate under conditions such that an aryl compound substituted with an aryl[d][1,2]iodaoxol-3-one group is formed.

In certain embodiments, contacting an aryl compound substituted with trifluoroborate and aryl[d][1,2]iodaoxol-3-one substituted at the iodine further comprises a silver salt. In certain embodiments, the silver salt is silver nitrate. In certain embodiments, contacting an aryl compound substituted with trifluoroborate and aryl[d][1,2]iodaoxol-3-one substituted at the iodine is in an acetonitrile or dichloromethane solvent. In certain embodiments, the contacting an aryl compound substituted with trifluoroborate and aryl[d][1,2]iodaoxol-3-one substituted at the iodine is at a temperature of less than 100 degrees Celsius.

In certain embodiments, this disclosure relates to methods of forming an aryl compound substituted with an aryl[d][1,2]iodaoxol-3-one group, e.g., for further use in producing a halogenated, fluorinated, or ¹⁸F aromatic compound, comprising contacting an aryl compound substituted with a trialkylsilane and aryl[d][1,2]iodaoxol-3-one substituted at the iodine with a halogen or acetate under conditions such that an aryl compound substituted with an aryl[d][1,2]iodaoxol-3-one group is formed.

In certain embodiments, contacting an aryl compound substituted with trialkylsilane and aryl[d][1,2]iodaoxol-3-one substituted at the iodine further comprises a silver salt. In certain embodiments, the silver salt is silver nitrate. In certain embodiments, contacting an aryl compound substituted with trialkylsilane and aryl[d][1,2]iodaoxol-3-one substituted at the iodine is in an acetonitrile or dichloromethane solvent. In certain embodiments, the contacting an aryl compound substituted with trialkylsilane and aryl[d][1,2]iodaoxol-3-one substituted at the iodine is at a temperature of less than 100 degrees Celsius.

### Imaging Methods

In certain embodiments, this disclosure relates to methods of imaging a radionuclide reported herein comprising administering the radionuclide to a subject or in an area of the subject and detecting the radionuclide at a location or the area in the subject. In certain embodiments, methods further comprise recording the location(s) or areas of the radionuclide on a non-transitory computer readable medium. In certain embodiments, methods further comprise creating an image based on locations of the radionuclide in the area and recording the image on a non-transitory computer readable medium.

In certain embodiments, this disclosure relates to methods comprising a) administering a composition comprising the tracer compound disclosed herein isotopically enriched with fluorine 18 to a subject; and scanning the subject for emissions from an area of the subject. In certain embodiments, the tracer binds to or enters cells, tissues, or organs. In certain embodiments, the emissions are from, on the exterior, or inside a brain, a cancerous, neuronal, cardiac, inflamed cell or tissue.

In certain embodiments, the tracer is administered at a dose of 0.04 to 0.9 μmol/kg or 0.04 to 10 μmol/kg. In certain embodiments, scanning the subject for emissions is for between 30 and 15 min. In certain embodiments, administering is at a dose of 0.9 μmol/kg and scanning the subject for emissions is for 15 min or more.

In certain embodiments, methods further comprise the step of detecting and/or measuring the emissions and creating an image indicating or highlighting the location of the compound isotopically enriched with fluorine 18 in the subject. In certain embodiments, the emissions are from, on the exterior, or inside a skull, brain, cancerous, neuronal, cardiac, inflamed cell or tissue.

In certain embodiments, methods comprise the step of detecting, measuring, and/or quantifying the emission providing an emission quantity and optionally correlating the emission measurement/detection/quantity to a concentration of tracer on or in the cells, tissue, tumor, or organ.

In certain embodiments, methods further comprise the step of correlating a low, high, or abnormal measurement, quantity, or concentration of tracer to the existence of or diagnosis of a subject having or at risk of being an abnormal neuron, cardiac, inflamed or cancer cell or tissue, or tumor or other target associated condition.

In certain embodiments, this disclosure relates to method of making the tracer comprising contacting a precursor compound or derivative with an isotopically enriched fluorine 18 negative ion producing the PET imaging tracer.

In certain embodiments, the reaction temperature is about or above 110 degrees Celsius. In certain embodiments, the reaction temperature is about or above 120 degrees Celsius. In certain embodiments, the reaction temperature is about or above 130 degrees Celsius. In certain embodiments, the reaction temperature is about or above 140 degrees Celsius. In certain embodiments, the reaction temperature is about or above 150 degrees Celsius. In certain embodiments, the reaction temperature is about or above 160 degrees Celsius. In certain embodiments, the reaction temperature is about or above 170 degrees Celsius.

In certain embodiments, the enriched fluorine 18 negative ion is a fluorine 18 potassium salt bound to a cryptand. In certain embodiments, the enriched fluorine 18 negative ion is a tetraalkylammonium base salt such as a tetrabutylammonium (TBA) fluorine 18 negative ion salt.

### Pharmaceutical Compositions and Kits

In certain embodiments, the disclosure contemplates pharmaceutical compositions comprising compounds disclosed herein or a pharmaceutically acceptable salt and optionally a pharmaceutically acceptable excipient.

In certain embodiments, this disclosure relates to kits comprising a precursor PET imaging compound. In certain embodiments, the precursor compound is an aryl compound substituted with an aryl[d][1,2]iodaoxol-3-one group disclosed herein. In certain embodiments, this disclosure relates to kits comprising a precursor PET imaging compound disclosed herein and potassium ion bound to a cryptand. In certain embodiments, this disclosure relates to kits aryl compound substituted with trialkyltin, trichloroborate, or trialkylsilane, and aryl[d][1,2]iodaoxol-3-one substituted at the iodine with a halogen or acetate or salt. In certain embodiments, the instructions provide for the activity at the end of synthesis. In certain embodiments, the kit provides instructions for use, e.g., wherein the instructions provide for the half-life of the radionuclide. In certain embodiments, the instructions provide that injection should be used within limited time from the time of the end of synthesis. In certain embodiments, the container is a sealed container such as a septum capped vial.

In certain embodiments, this disclosure relates to kits comprising the precursor compound or derivatives and starting materials to make the radionuclide tracer, and/or a substance for preparing a radionuclide in a cyclotron. In certain embodiments, the kit comprises a container having water, H₂¹⁸O, and/or ethanol in water solution. In certain embodiments, the container is sealed from the atmosphere. In certain embodiments, kits comprise a solid support or filter. In certain embodiments, the filter may be used to purify a radionuclide tracer disclosed herein.

In some embodiments, the solid support is selected from the group of solid phase extraction resins or liquid chromatography resins, e.g., silica (oxide) based or non-silica (metal oxide or polymers) based particles optionally functionalized (e.g., by organosilanization) with alkyl chains for example C4, C8, C18, C30 or other functional groups, e.g., polar groups (amide, carbamate, and urea) embedded within alkyl chains or branched alkyl groups or polymeric packings.

In some embodiments, the solid support is selected from the group consisting of solid phase extraction resins and liquid chromatography resins resulting from the copolymerization of divinylbenzene and/or styrene, or by the copolymerization with vinylpyrrolidone, vinyl acetate, (methacryloyloxymethyl)naphthalene, 4,4′-bis(maleimido)diphenylmethane, p,p′-dihydroxy diphenylmethane diglycidyl methacrylic ester, p,p′-dihydroxydiphenylpropane diglycidyl methacrylic ester, 2-hydroxyethylmethacrylate (HEMA), 2,2-dimethylaminoethylmethacrylate (DMAEMA), ethylene dimethacrylate glycidyl methacrylate, N-vinyl carbazole, acrylonitrile, vinyl pyridine, N-methyl-N-vinyl acetamide, amino styrene, methylacrylate, ethylacrylate, methyl methacrylate, N-vinyl caprolactam, N-methyl-N-vinyl acetamide.

In some embodiments, the solid support comprises or is functionalized with or preconditioned with:
quaternary ammonium salts, e.g., tetraethylammonium carbonate, tetrabutylammonium carbonate;
potassium carbonate cryptands such as [2.2.2] cryptand N(CH₂CH₂OCH₂CH₂OCH₂CH₂)₃N, 1,4,10-trioxa-7,13-diaza-cyclopentadecane, 4,7,13,16,21,24-hexaoxa-1,10-diazabicyclo[8.8.8]hexacosane, 4,7,13,16,21-pentaoxa-1,10-diazabicyclo[8.8.5]tricosane, 4,7,13,18-tetraoxa-1,10-diazabicyclo[8.5.5] eicosane, 5,6-benzo-4,7,13,16,21,24-hexaoxa-1,10-diazabicyclo[8.8.8]hexacos-5-ene;
crown ethers such as 4′-aminobenzo-15-crown-5, 4′-aminobenzo-15-crown-5, 4′-aminobenzo-15-crown-5 hydrochloride, 4′-aminobenzo-18-crown-6, a′-Aminodibenzo-18-crown-6, 2-aminomethyl-15-crown-5, 2-aminomethyl-15-crown-5, 2-aminomethyl-18-crown-6, 4′-amino-5′-nitrobenzo-15-crown-5, 4′-amino-5′-nitrobenzo-15-crown-5, 1-aza-12-crown-4, 1-aza-15-crown-5, 1-aza-15-crown-5, 1-aza-18-crown-6, 1-aza-18-crown-6, benzo-12-crown-4, 5,6-benzo-4,7,13,16,21,24-hexaoxa-1,10-diazabicyclo[8.8.8]hexacos-5-ene, 1-benzyl-1-aza-12-crown-4, bis[(benzo-15-crown-5)-15-ylmethyl]pimelate, 4′-bromobenzo-15-crown-5, 4-tert-butylbenzo-15-crown-5, 4-tert-butylcyclohexano-15-crown-5, 4′-carboxybenzo-15-crown-5;
calixarenes such as 4-tert-butylcalix[4]arene, 4-tert-butylcalix[4]arene, 4-tert-butylcalix[4]arene, 4-tert-butylcalix[5]arene, 4-tert-butylcalix[6]arene, 4-tert-butylcalix[6]arene, 4-tert-butylcalix[6]arene, 4-tert-butylcalix[8]arene, 4-tert-butylcalix[8]arene, 4-tert-butylcalix[4]arene-tetraacetic acid tetraethyl ester, 4-tert-butylcalix[4]arene tetraacetic acid tetraethyl ester, 4-tert-butylcalix[4]arene-tetraacetic acid triethyl ester, calix[4]arene, calix[6]arene, calix[8]arene, 4-(chloromethyl)calix[4]arene, 4-isopropylcalix[4]arene, C-methylcalix[4]resorcinarene, C-methylcalix[4]resorcinarene, meso-octamethylcalix(4)pyrrole, 4-sulfocalix[4]arene, 4-sulfocalix[4]arene sodium salt, C-undecylcalix[4]resorcinarene monohydrate, C-undecylcalix[4]resorcinarene monohydrate;
cyclodextrins such as α-cyclodextrin, β-cyclodextrin, γ-cyclodextrin, (2,6-di-O-)ethyl-β-cyclodextrin, 6-O-α-D-glucosyl-β-cyclodextrin, heptakis(6-O-t-butyldimethylsilyl-2,3-di-O-acetyl)-β-cyclodextrin, heptakis(2,6-di-O-methyl)-β-cyclodextrin, heptakis(2,3,6-tri-O-acetyl)-β-cyclodextrin, heptakis(2,3,6-tri-O-benzoyl)-β-cyclodextrin, hexakis (6-O-tertbutyl-dimethylsilyl)-α-cyclodextrin, hexakis (2,3,6-tri-O-acetyl)-α-cyclodextrin, hexakis (2,3,6-tri-O-methyl)-α-cyclodextrin, (2-hydroxyethyl)-β-cyclodextrin, 6-O-α-maltosyl-β-cyclodextrin hydrate, methyl-β-cyclodextrin, 6-monodeoxy-6-monoamino-β-cyclodextrin, octakis (6-O-t-butyldimethylsilyl)-γ-cyclodextrin, sulfopropyl-β-cyclodextrin, triacetyl-α-cyclodextrin, triacetyl-β-cyclodextrin;
EDTA and derivatives such as ethylenediamine-N,N′-diacetic acid, 2-bis(2-aminophenoxy)ethane-N,N,N′,N′-tetraacetic acid, trans-1,2-diaminocyclohexane-N,N,N′,N′-tetraacetic acid monohydrate, trans-1,2-diaminocyclohexane-N,N,N′,N′-tetraacetic acid monohydrate, 1,3-diamino-2-hydroxypropane-N,N,N′,N′-tetraacetic acid, 1,2-diaminopropane-N,N,N′,N′-tetraacetic acid, 1,3-diaminopropane-N,N,N′,N′-tetraacetic acid, 1,3-diamino-2-propanol-N,N,N′,N′-tetraacetic acid, diethylenetriamine-pentaacetic acid calcium trisodium salt hydrate, N-(2-hydroxyethyl)ethylenediamine triacetic acid trisodium salt hydrate, N-(2-hydroxyethyl)ethylenediamine-N,N′,N′-triacetic acid; and/or
polyethylene glycols (PEG), polyethylene oxides (PEO).

### Synthesis of 18F-Fluoroarenes Through Radiofluorination of 1-Arylbenziodoxolones

Disclosed herein are methods for aromatic ¹⁸F-labeling through ¹⁸F-fluorination of 1-arylbenziodoxolones. 1-Arylbenziodoxolones are hypervalent iodine-based precursors for ¹⁸F-labeling of arenes which can be synthesized from aryl trifluoroborates, aryl silanes, or aryl stannanes under mild, one-step conditions. The method produces both electron-rich and electron-deficient ¹⁸F-labeled aryl fluorides with desirable radiochemical yields. Experiments indicate functional group compatibility, including substrates with oxidation-sensitive functional groups, like aryl alkenes, alkynes, iodides, aldehydes, and sulfoxides. The versatility of this method is further demonstrated by its application to a range of clinically relevant PET ligands, including a fibroblast activation protein targeted ligand, [¹⁸F] compound 8, and a metabotropic glutamate receptor 5 ligand, [¹⁸F]FPEB.

Positron emission tomography (PET) is a medical imaging technique useful for investigating biological processes in vivo by detecting signals from exogenous radioactive probes.

PET allows one to monitor disease progression and assess drug efficacy stem from, in part, innovations in positron emitter-labeling methods and the development of novel PET tracers, especially those involving ¹⁸F. The clean positron emission (97% β-decay ratio), appropriate half-life (109.7 min), low maximum positron energy (0.635 MeV), high resolution (0.6 mm), and its scalable production with high molar activity make ¹⁸F an attractive positron-emitting radionuclide in PET. PET tracers can be used in pre-clinical and clinical research; thus, introducing ¹⁸F into target molecules, particularly in the form of ¹⁸F-labeled aryl fluorides provided enhanced stability against the defluorination process in vivo.

Nucleophilic aromatic substitution with [¹⁸F]fluoride ion is a method for producing ¹⁸F-fluoroarenes. One can use activated arenes with a leaving group (—N+Me₃, —S+Me₂, —NO₂, halogens, mesylate, tosylate, or triflate, etc.), triarylsulfonium salts, diaryl sulfoxides, and N-arylsydnones. By using transition-metal-mediated ¹⁸F-fluorination it is contemplated that one can improve reactivity, selectivity, and functional group tolerance when preparing ¹⁸F-fluoroarenes.

Reported herein are method for synthesizing ¹⁸F-fluoroarenes through radiofluorination of 1-arylbenziodoxolones, along with approaches to obtain the corresponding labeling precursors from readily available aryl trifluoroborates, aryl silanes, or aryl stannanes under oxidation-free conditions in an efficient manner.

1-Aryl-benziodoxolones can be synthesized from electron-rich aromatics and 2-iodobenzoic acid in oxidizing conditions (e.g. oxone and conc. H₂SO₄). Functional group tolerance to these conditions sometimes limits the substrate scope of 1-arylbenziodoxolones. A Lewis acid-catalyzed iodonium transfer from acetoxy-benziodoxolones to electron-rich aromatics is a milder alternative. Indoles and pyrroles sometimes present challenges in controlling regioselectivity. Contemplated herein are methods for synthesizing 1-aryl-benziodoxolones from aryl trifluoroborates, aryl silanes, or aryl stannanes. These methods enable the preparation of 1-aryl-benziodoxolones under mild conditions with providing functional group tolerance and larger substrate scope.

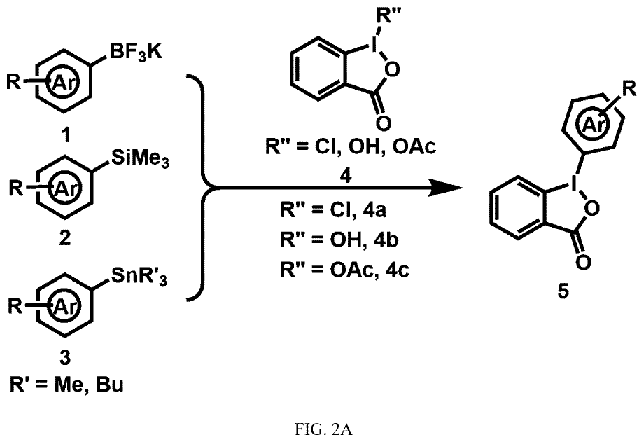

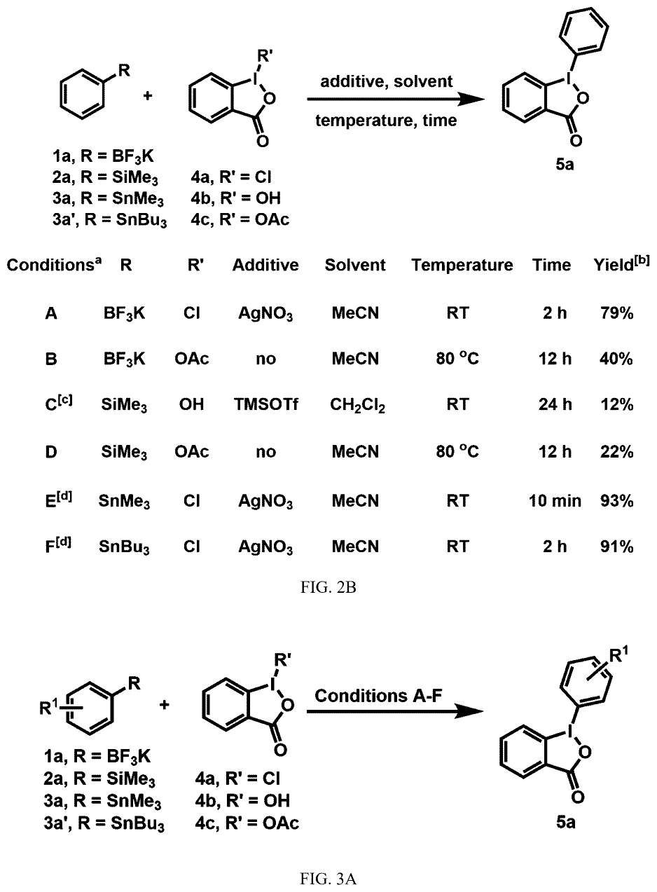

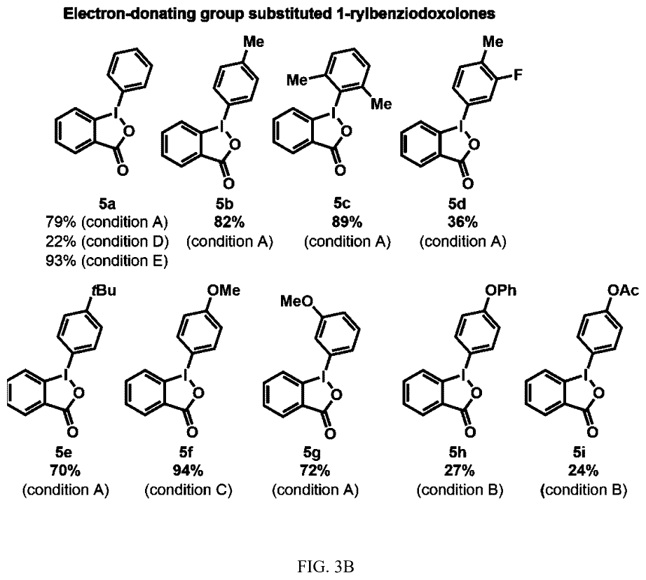

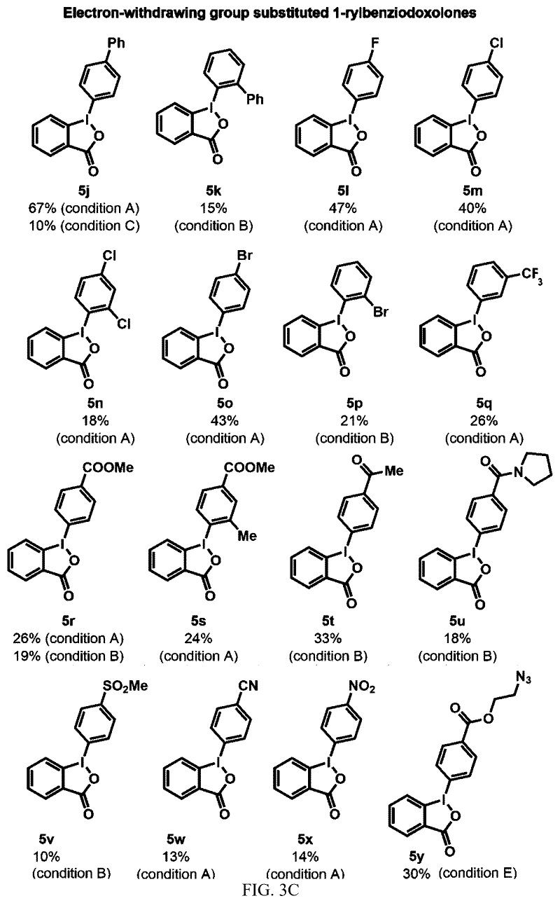

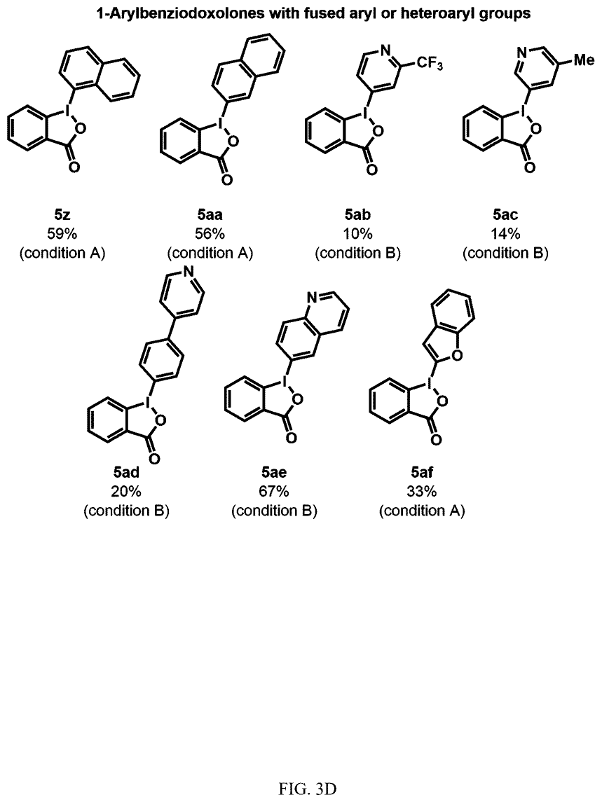

For the synthesis of 1-arylbenziodoxolones, the synthesis of 5a from potassium phenyltrifluoroborate 1a, trimethyl(phenyl)silane 2a, trimethyl(phenyl)stannane 3a, and tributyl(phenyl)stannane 3a′ were investigated (FIG. 2B). Starting from phenyltrifluoroborate 1a, 5a was prepared using λ3-iodane 4a in the presence of silver nitrate as the additive in 79% yield or using λ3-iodane 4c in 40% yield. When using trimethyl(phenyl)silane 2a, 5a was obtained with λ3-iodane 4b in the presence of trimethylsilyl trifluoromethanesulfonate as the additive in 12% yield or with λ3-iodane 4c in 22% yield. From trimethyl(phenyl)stannane 3a and tributyl(phenyl)stannane 3a′, 5a was synthesized with λ3-iodane 4a in the presence of silver nitrate as the additive in 93% and 91% yields, respectively.

The substrate scope of 1-arylbenziodoxolones 5 was investigated. A wide range of substituents on the aryl group appears to be compatible with the discovered synthetic conditions, including electron-donating groups, such as methyl (-Me, 5b-5d), tert-butyl (-tBu, 5e), methoxy (—OMe, 5f and 5g), phenoxy (—OPh, 5h), and acetoxy (—OAc, 5i), as well as electron-withdrawing groups like phenyl (-Ph, 5j and 5k), halides (—F, —Cl, and —Br, 5l-5p), trifluoromethyl (—CF3, 5q), methoxycarbonyl (—CO₂Me, 5r and 5s), acetyl (—Ac, 5t), pyrrolidine-1-carbonyl (5u), methylsulfonyl (—SO₂Me, 5v), cyano (—CN, 5w), nitro (—NO₂, 5x), and 2-azidoethoxycarbonyl (—CO₂CH₂CH₂N₃, 5y). These derivatives (products 5) were obtained in yields ranging from 10 to 94%. Furthermore, fused aryl (5z and 5aa) and heteroaryl (5ab-5af) benziodoxolones were synthesized in 10-67% yields in similar conditions. Notably, this method was extended to the synthesis of 1-arylbenziodoxolones 5 bearing with oxidation-sensitive functional groups, such as alkynyl (5ag), vinyl (5ah), iodo (—I, 5ai-5ak), formyl (—CHO, 5al), and methylsulfinyl (—SOMe, 5am). These compounds would be challenging or inefficient to prepare under oxidative conditions using previous methods. Fortunately, ligand exchange between heterocyclic λ3-iodanes 4 and the corresponding aryl trifluoroborates 1, aryl silanes 2, or aryl stannanes 3 under mild conditions enable the synthesis of a variety of well functionalized 1-arylbenziodoxolone precursors 5 for ¹⁸F-fluorination.

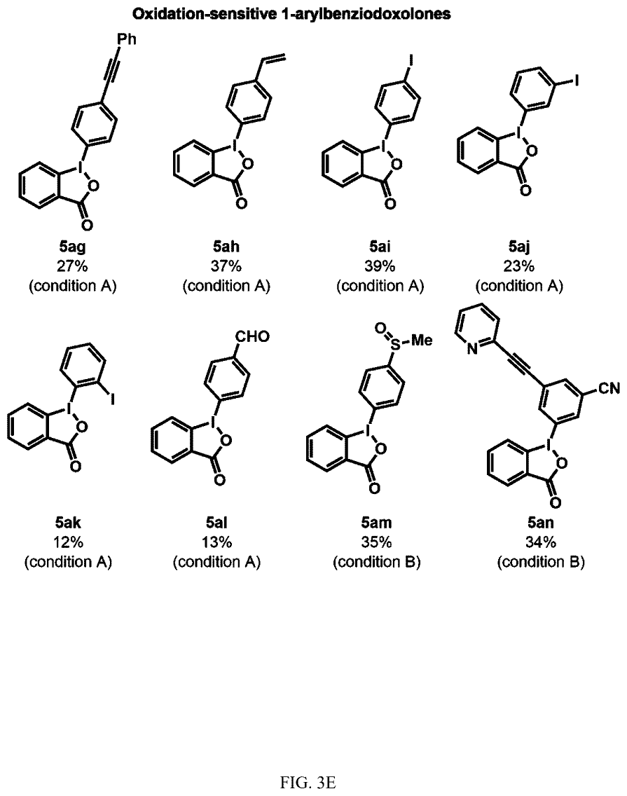

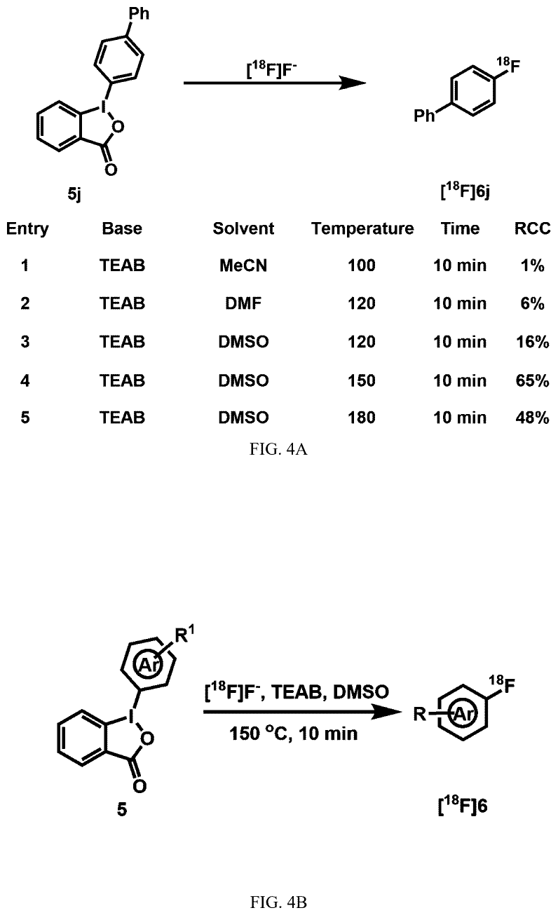

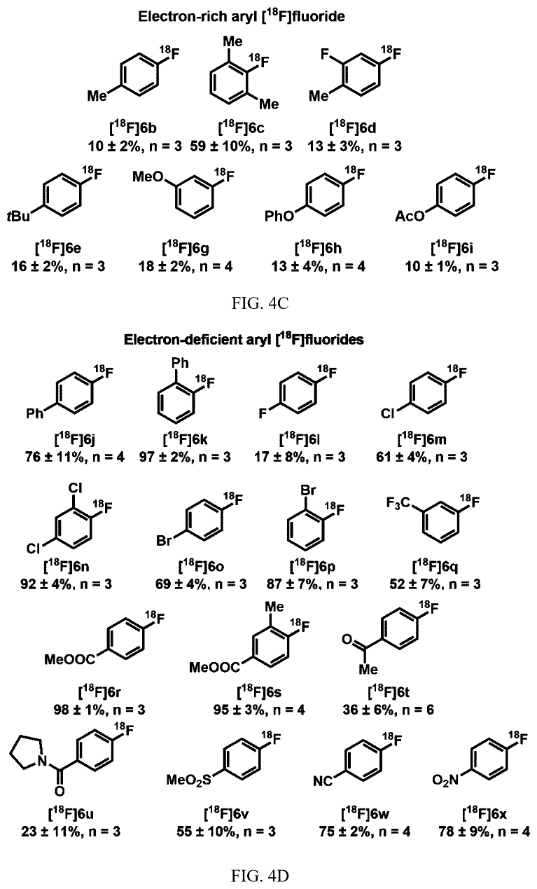

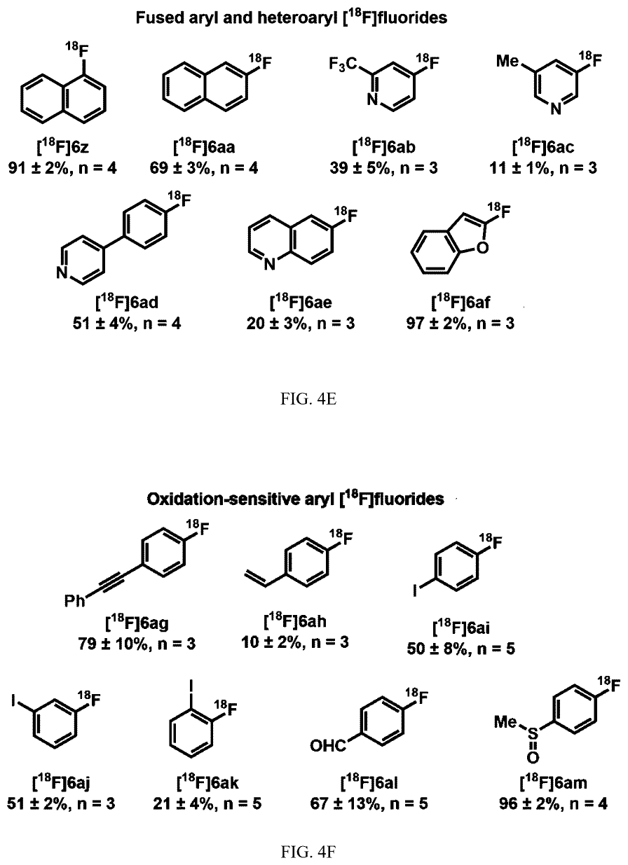

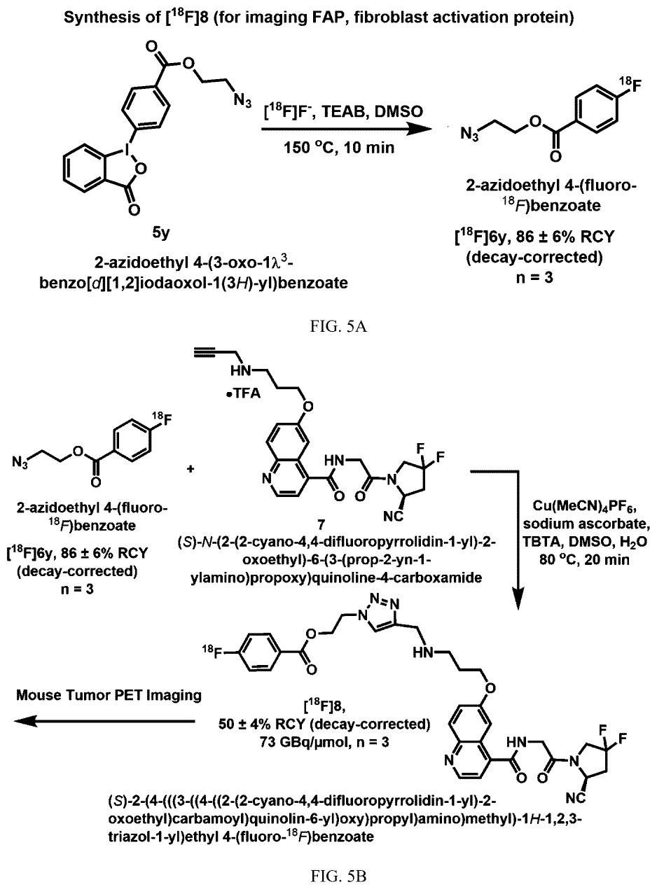

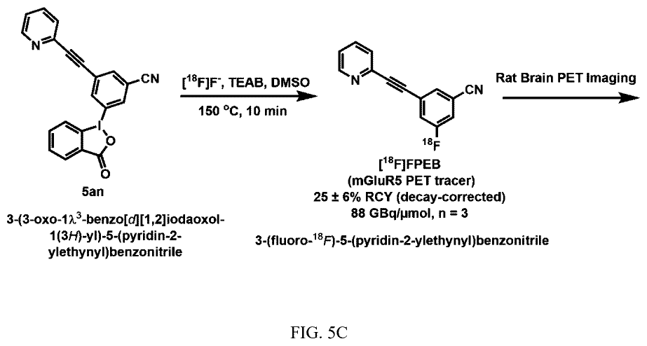

Using 1-arylbenziodoxolones ¹⁸F-labeling conditions were investigated (See FIG. 4A). Compound 5j was labeled with [¹⁸F]fluoride using Bu₄NHCO₃ (TEAB) as the base and MeCN (acetonitrile) as the solvent at 100° C. for 10 min (entry 1). Under these conditions, the desired product [18F]6j was formed in 1% RCC (radiochemical conversion). A survey of solvents and temperature effect showed that polar solvents and higher temperatures were beneficial to this reaction (entries 1-5). With DMSO as the optimal solvent, ¹⁸F-labeling reactions of 5j were conducted with different bases and the results showed that Bu₄NHCO₃ (TEAB) was the most effective base. Optimization of several reaction parameters revealed that product [¹⁸F]6j could be obtained in 65% RCC with Bu₄NHCO₃ (TEAB) as the base, and DMSO as solvent at 150° C. for 10 min (entry 4). Under these conditions, by-product 2-[¹⁸F]fluorobenzoic acid was also observed in 6% RCC.

To further optimize labeling efficiency, the auxiliary benziodoxolone was modified to examine the effects of the substituent in the ¹⁸F-labeling reactions. The introduction of electron-donating groups, such as —OMe or -Me, into the auxiliary benziodoxolone suppressed the formation of ¹⁸F-labeled substituted benzoic acid, and the RCC of the desired product [¹⁸F]6j also decreased. Conversely, electron-withdrawing groups, such as —CF₃ or —CN, led to the predominance of ¹⁸F-labeled substituted benzoic acid over the desired product [¹⁸F]6j. To balance the efficiency and regioselectivity of ¹⁸F-labeling, non-substituted benziodoxolone was chose as the auxiliary in further investigation of the reaction scope. Notably, the minor by-product, ¹⁸F-labeled substituted benzoic acid, is water soluble and always in the solvent front during reverse phase HPLC purification.

With the optimized reaction conditions established, the scope of the ¹⁸F-fluorination reaction was explored using a variety of structurally diverse 1-arylbenziodoxolones 5. This method effectively delivers various substituted aryl [¹⁸F]fluorides [¹⁸F]6. Electron-donating groups, such as methyl (-Me, [¹⁸F]6b-[¹⁸F]6d), tert-butyl (-tBu, [¹⁸F]6e), methoxy (—OMe, [¹⁸F]6g), phenoxy (—OPh, [¹⁸F]6h), and acetoxy (—OAc, [¹⁸F]6i), were successfully incorporated into the aryl ring. Similarly, aryl [¹⁸F]fluorides [¹⁸F]6 with electron-withdrawing groups, including phenyl (-Ph, [¹⁸F]6j and [¹⁸F]6k), halides (—F, —Cl, and —Br, [¹⁸F]6l-[¹⁸F]6p), trifluoromethyl (—CF₃, [¹⁸F]6q), methoxycarbonyl (—COOMe, [¹⁸F]6r and [¹⁸F]6s), acetyl (—Ac, [¹⁸F]6t), pyrrolidine-1-carbonyl ([¹⁸F]6u), methylsulfonyl (—SO₂Me, [¹⁸F]6v), cyano (—CN, [¹⁸F]6w), and nitro (—NO₂, [¹⁸F]6x), were also prepared with high RCCs. Furthermore, fused aryl [¹⁸F]fluorides ([¹⁸F]6z and [¹⁸F]6aa) and heteroaryl [¹⁸F]fluorides ([¹⁸F]6ab-[¹⁸F]6af) were also compatible and can be prepared. Notably, this method translated to the syntheses of aryl [¹⁸F]fluorides [¹⁸F]6 with oxidation-sensitive functional groups, including alkynyl ([¹⁸F]6ag), vinyl ([¹⁸F]6ah), iodo (—I, [¹⁸F]6ai-[¹⁸F]6ak), formyl (—CHO, [¹⁸F]6al), and methylsulfinyl (—SOMe, [¹⁸F]6am).

### Radiosynthesis of a Fibroblast Activation Protein (FAP) PET Ligand, [¹⁸F]8, for Oncology PET Imaging

The practical utility of ¹⁸F-fluorination of 1-arylbenziodoxolones was applied to the radiosynthesis of a fibroblast activation protein (FAP) PET ligand, [¹⁸F]8, for oncology PET imaging and a metabotropic glutamate receptor (mGluR5) PET tracer, [¹⁸F]FPEB for neurology applications. Because of the high expression in tumor cells, FAP has become one of the most promising targets in the diagnosis of most types of cancers by PET imaging. This ¹⁸F-fluorination method was utilized to ¹⁸F-label the clickable azide, which could conjugate with the FAP-targeting vector and thus rapidly expand the FAP PET ligand library. Using the discovered conditions, the ¹⁸F-labeled azide [¹⁸F]6y was obtained in 86% decay-corrected RCY. The azide [¹⁸F]6y was then conjugated with alkyne 7 to afford FAP PET ligand [18F]8 (FAP IC₅₀=0.79 nM) in 50% RCY and a molar activity of 73 GBq/μmol (1.97 Ci/μmol). PET imaging of [¹⁸F]8 in tumor-bearing mice demonstrated high tumor uptake and excellent FAP-binding specificity in vivo, as confirmed by baseline and blocking studies with a known FAP inhibitor FAPI-46. Because of the expression and functions of mGluR5 in different neuronal processes, PET imaging of mGluR5 is helpful in investigating its role in various neuropsychiatric disorders and neurodegenerative diseases, including depression, post-traumatic stress disorder (PTSD), anxiety, schizophrenia, Alzheimer's disease (AD), Parkinson's disease (PD), and Huntington's disease (HD). The most widely used mGluR5 PET tracer [¹⁸F]FPEB was synthesized using this method in 25% RCY with a molar activity of 88 GBq/μmol (2.38 Ci/μmol). PET imaging of rat brains with [¹⁸F]FPEB revealed high brain uptake, heterogenous distribution and excellent mGluR5-binding specificity in vivo.

1. A method of forming a fluorinated aromatic compound comprising contacting an aryl compound substituted with an aryl[d][1,2]iodaoxol-3-one group and a fluoro ion under conditions providing a fluorinated aromatic compound with a fluorine in place of the aryl[d][1,2]iodaoxol-3-one group.

2. The method of claim 1, wherein the fluoro ion is isotopically enriched with fluorine 18 isotope.

3. The method of claim 1, wherein the aryl[d][1,2]iodaoxol-3-one group is a benzo[d][1,2]iodaoxol-3-one group optionally substituted.

4. The method of claim 1, wherein the conditions include the presence of a base in a solvent at a temperature above 100 degrees Celsius.

5. The method of a claim 4, wherein the base is a quaternary ammonium base.

6. The method of a claim 5, wherein the quaternary ammonium base is a tetra-alkyl ammonium salt.

7. The method of a claim 6, wherein the tetra-alkyl ammonium salt is tetraethylammonium bromide (TEAB).

8. The method of claim 4, wherein the solvent is dimethyl sulfoxide (DMSO).

9. The method of claim 4, wherein the temperature is above 120 degrees Celsius.

10. The method of claim 4, wherein the temperature is above 140 degrees Celsius.

11. A method of forming an aryl compound substituted with an aryl[d][1,2]iodaoxol-3-one group comprising contacting an aryl compound substituted with trialkyltin and aryl[d][1,2]iodaoxol-3-one substituted at the iodine with a halogen or acetate under conditions such that an aryl compound substituted with an aryl[d][1,2]iodaoxol-3-one group is formed.

12. The method of claim 11, wherein contacting an aryl compound substituted with trialkyltin and aryl[d][1,2]iodaoxol-3-one substituted at the iodine further comprises a silver salt.

13. The method of claim 12, wherein the silver salt is silver nitrate.

14. The method of claim 13, wherein contacting an aryl compound substituted with trialkyltin and aryl[d][1,2]iodaoxol-3-one substituted at the iodine is in an acetonitrile solvent.

15. A compound 2-azidoethyl 4-(fluoro-¹⁸F)benzoate.
